(19) United States
(12) Patent Application Publication
(10) Pub. No.: US 2006/0276373 A1
(43) Pub. Date: Dec. 7, 2006

(54) Neuroprotective complex for treatment of cerebral ischemia and injury

(76) Inventors: Nicolas G. Bazan, New Orleans, LA (US); Myron D. Ginsberg, Coconut Grove, FL (US); James P. Moises, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/374,724

(22) Filed: Mar. 13, 2006

Related U.S. Application Data
(63) Continuation-in-part of application No. 11/234,715, filed on Sep. 23, 2005.
Application No. 11/234,715 is a division of application No. 10/763,698, filed on Jan. 23, 2004, now abandoned.

(51) Int. Cl. A61K 38/38; A61K 31/202

(52) U.S. Cl. 514/2; 514/560

(57) ABSTRACT

The invention provides a pharmaceutical composition useful in treating cerebral ischemia and traumatic cerebral injury. The pharmaceutical composition is also useful as a prophylactic treatment during surgical procedures wherein the potential for ischemic tissue damage is present. Also included in the invention is a method for preparing the pharmaceutical composition, as well as methods for treatment.

### STATEMENT REGARDING CONTINUITY

This is a continuation-in-part of co-pending application Ser. No. 11/234,715 filed on Sep. 23, 2005, which was a divisional of co-pending application Ser. No. 10/763,698, filed Jan. 23, 2004.

### STATEMENT REGARDING FEDERALLY SPONSORED RESEARCH OR DEVELOPMENT

The development of this invention was partially funded by the United States Government under grant HR0011-04-C-0068, from the Defense Advanced Research Projects Agency of the Department of Defense. The United States Government has certain rights in this invention.

### FIELD OF THE INVENTION

The present invention relates to a neuroprotective complex of human albumin and polyunsaturated fatty acid, particularly docosahexaenoic acid, that is useful in treating ischemic stroke, as well as other types of injuries, such as traumatic brain, eye and spinal cord injury, that may produce ischemic or traumatic tissue damage, and during surgical procedures such as carotid endarterectomy and coronary bypass surgery, where the potential for ischemic tissue damage is present.

### BACKGROUND OF THE INVENTION

Stroke is characterized by the sudden loss of circulation to an area of the brain, resulting in a corresponding loss of neurologic function. Also referred to as cerebrovascular accident or stroke syndrome, stroke is a nonspecific term encompassing a heterogeneous group of pathophysiologic causes, including thrombosis, embolism, and hemorrhage. Acute ischemic stroke refers to strokes caused by thrombosis or embolism and accounts for over 80% of all strokes.

The fundamental hypothesis in stroke research is that ischemia produces disability and death by initiating a cascade of cellular processes that eventually lead to neuronal death. The cascade begins rapidly after ischemia, when a thrombus or embolus from the heart, aorta, or carotid or vertebral arteries lodges in the intracranial circulation of the brain, blocking blood flow to the distal portion of the affected vessel. The processes involved in stroke injury at the cellular level are referred to as the ischemic cascade. Within seconds to minutes of the loss of glucose and oxygen delivery to neurons, the cellular ischemic cascade begins. This is a complex process that begins with cessation of the electrophysiologic function of the cells.

As the process continues, the cell metabolism changes from aerobic to anaerobic. With the depletion of ATP stores, membrane ion pumps fail, leading to increased intracellular concentration of sodium and calcium. The cell begins to experience injury from calcium-mediated cytotoxic reactions and release of excitatory neurotransmitters, specifically glutamate. These processes lead to activation of proteases, endonucleases, phospholipases, and nitric oxide synthase and formation of free radicals. The resultant neuronal and glial injury produces edema in the ensuing hours to days after stroke, causing further injury to the surrounding neuronal tissues.

An acute vascular occlusion produces heterogeneous regions of ischemia in the dependent vascular territory. The quantity of local blood flow comprises any residual flow in the major arterial source and collateral supply, if any. Regions of the brain without significant flow are referred to collectively as the core, and these cells are presumed to die within minutes. Zones of decreased or marginal perfusion are collectively called the ischemic penumbra. Tissue in the penumbra can remain viable for several hours, and pharmacologic interventions for preservation of neuronal tissue target the penumbra.

Drug therapies have been investigated, which, if administered after the onset of acute stroke, may potentially succeed in diminishing the extent of tissue damage and improving functional outcome. The proposed drug therapies are based on laboratory investigations of cerebral ischemia that have identified key biochemical and molecular mechanisms, including the central roles of excitotoxicity, tissue calcium overload, oxygen radicals, inflammatory mediators, and other factors, that contribute to the death of brain tissue.

Hemodiluting agents have been widely investigated as a potential therapy for ischemic stroke. The primary rationale for this approach is that cerebral blood flow varies inversely with hematocrit and whole-blood viscosity, and hemodilution has been shown to increase cerebral blood flow of both the normal and ischemic brain, either by decreasing blood viscosity or by vasodilation in response to diminished oxygen delivery. Albumin, an endogenous plasma protein, is commonly regarded as a hemodiluting agent. Importantly, albumin is an evolutionarily highly conserved molecule that subserves numerous vital physiological functions. Among these are fatty-acid transport, antioxidant function, maintenance of vascular endothelium, and oncotic activity. All of these functions are relevant to albumin's neuroprotective effect.

Several studies have reported a positive effect in reducing ischemic brain injury, including diminished brain edema and infarct volume, in rats with middle cerebral artery occlusion (MCAO) treated with high doses of albumin administered shortly after the onset of ischemia. Albumin has also been demonstrated to reduce the volume of contusion-injury in animals subjected to brain trauma. While albumin administration in humans has been found to be generally well tolerated, several adverse reactions may occur. When albumin is administered in high doses for the treatment of ischemia or other conditions, intravascular volume overload, congestive heart failure and pulmonary edema are the chief concerns. In rare circumstances, chills, fever, tachycardia, hypotension, urticaria, skin rash and nausea have been reported

As indicated above, high doses of human serum albumin, when administered intravenously within a therapeutic window extending up to four hours after the onset of MCAo, are highly neuroprotective reducing infarct volume and edema, thus improving neurological scores and protecting the ischemic penumbra. However, the effect of albumin therapy on local cerebral blood flow in areas that show histological neuroprotection is of lower magnitude than would be expected on the basis of its marked neuroprotectant effect. This suggests that other, non-hemodynamic mechanisms contribute to albumin-mediated neuroprotection.

In addition to albumin's neuroprotective characteristics, the protein is known to have several multifaceted intravascular effects. Albumin is a specific inhibitor of endothelial cell apoptosis. Several albumin-binding proteins have been identified on endothelial cells from many origins, including brain, that mediate its transcytosis and endocytosis. Albumin also constitutes a major antioxidant defense against oxidizing agents generated both by endogenous processes (such as neutrophil myeloperoxidase) and by exogenous compounds. Albumin also plays a crucial role in the transport of fatty acids and in the binding of metabolites and drugs. After considerable research, the inventors herein have discovered that albumin's role in the transport mechanism of fatty acids influences its neuroprotective effect.

The omega-3 fatty acid, docosahexaenoic acid (22:6, n-3, DHA), is highly concentrated in synapses, is required during development and for synaptic plasticity, and participates in neuroprotection. Free DHA is released through phospholipases from membrane phospholipids in response to seizures. See, N. G. Bazan, “Effects of ischemia and electroconvulsive shock on free fatty acid pool in the brain,” Biochim. Biophys. Acta, vol. 218, pp. 1-10 (1970); and D. L. Birkle et al., “Effect of bicuculline-induced status epilepticus on prostaglandins and hydroxyeicosatetraenoic acids in rat brain subcellular fractions,” J. Neurochem., vol. 48, pp. 1768-1778 (1987). Recently the structure and bioactivity of neuroprotectin D1, a potent DHA-derived mediator in brain ischemia-reperfusion and in oxidative stress, has been described. See V. L. Marcheselli et al., “Novel docosanoids inhibit brain ischemia-reperfusion-mediated leukocyte infiltration and pro-inflammatory gene expression,” J. Biol. Chem., vol. 278, pp. 43807-817 (2003); and P. K. Mukherjee et al., “Neuroprotectin D1: A docosahexaenoic acid-derived docosatriene protects human retinal pigment epithelial cells from oxidative stress,” Proc. Natl. Acad. Sci., USA, vol. 101, pp. 8491-96 (2004). Several polyunsaturated fatty acids (PUFAs), including DHA, have been suggested to attenuate epileptic activity in in vitro studies on rat brain cells or hippocampal slices. See C. Young et al., “Docosahexaenoic acid inhibits synaptic transmission and epileptiform activity in the rat hippocampus,” Synapse, vol. 37, pp. 90-94 (2000); and Y. Xiao et al., “Polyunsaturated fatty acids modify mouse hippocampal neuronal excitability during excitotoxic or convulsant stimulation,” Brain Res., vol. 846, pp. 112-121 (1999).

DHA complexed to albumin has been shown to enhance neuroprotectin 1 synthesis in human retinal pigment epithelial cells, and to be strongly neuroprotective in a mouse model of brain ischemia. See P. K. Mukherjee et al., “Neuroprotectin D1: A docosahexaenoic acid-derived docosatriene protects human retinal pigment epithelial cells from oxidative stress,” Proc. Natl. Acad. Sci., U.S.A., vol. 101, pp. 8491-8496 (2004); and L. Belayev et al., “Docosahexaenoic acid complexed to albumin elicits high-grade ischemic neuroprotection,” Stroke, vol. 36, pp. 118-123 (2005).

Traumatic brain injury (TBI) triggers an inflammatory cascade that results in increased permeability of the blood brain barrier, edema of the brain, and posttraumatic neuronal cell death. See E. Csuka et al., “IL-10 levels in cerebrospinal fluid and serum of patients with severe traumatic brain injury: relationship to IL-6, TNF-alpha, TGF-beta 1, and blood-brain barrier function,” J. Neuroimmunol., vol. 101, pp. 211-221 (1999). One model to study TBI is the fluid percussion head injury model. See, for example, T. K. McIntosh et al., “Traumatic brain injury in the rat: characterization of a midline fluid-percussion model,” Cent. Nerv. Syst. Trauma, vol. 4, pp. 119-34 (1987). Human albumin has been shown to be neuroprotective in traumatic models of brain injury. See L. Belayev et al., “Posttreatment with high-dose albumin reduces histopathological damage and improves neurological deficit following fluid percussion brain injury in rats,” J. Neurotrauma, vol. 16, pp. 445-453 (1999); and M. Is et al., “Intraventricular albumin: an optional agent in experimental post-traumatic brain edema,” Neurol. Res., vol. 27, pp. 67-72 (2005). Omega-3 enriched dietary supplements, including DHA, were found to counteract the studied effects of mild fluid percussion injury in rats. See A. Wu et al., “Dietary omega-3 fatty acids normalize BDNF levels, reduce oxidative damage, and counteract learning disability after traumatic brain injury in rats,” J. Neurotrauma, vol. 21, pp. 1457-1467 (2004).

### SUMMARY OF THE INVENTION

The inventors herein have discovered that the albumin-mediated systemic mobilization and supply of free fatty acids to the brain, which favors the replenishment of polyunsaturated fatty acids lost from cellular membranes during ischemia and/or serve as an alternate energy source, contributes to albumin neuroprotection. More specifically, MCAo selectively activates the transport of docosapentaenoic acid (22:5n-3) and docosahexaenoic acid (22:6n-3, DHA). Studies have suggested that polyunsaturated fatty acids may have therapeutic value for cerebral pathologies as they block neuronal death by inhibiting glutamatergic transmission. Surprisingly, however, it has been discovered that albumin loaded with polyunsaturated fatty acids, particularly docosahexaenoic acid (22:6n-3, DHA), produces high-grade histologic and neurologic protection at a dose considerably below that required when administering albumin alone.

Besides the neuroprotective function of the disclosed invention, the DHA—Albumin complex can also be used as a prophylactic treatment during surgical procedures wherein the potential for ischemic tissue damage is present. Ischemia/Reperfusion injury is a major cause of tissue damage and death that occurs when blood flow to an organ is interrupted and then later re-established, which can occur during major vascular surgery or in other situations. The potential for ischemic tissue damage may be reduced by the administration of DHA-Albumin complex, particularly with regard to surgical procedures involving large blood vessels, for example, procedures for treating thoracic and abdominal aortic aneurysms. Other procedures wherein ischemic tissue damage may be prevented by employing the treatments disclosed herein include coronary artery bypass grafting, coronary angioplasty, implantation of arterial stints, mesenteric and renal reconstruction, infrainguinal procedures, carotid endarterectomy, venous surgery, and major vascular trauma reconstructions.

The administration of an albumin-docosahexaenoic acid (DHA) complex decreased the degree of brain edema resulting from a traumatic brain injury. This was shown using a fluid percussion head injury (FPI) model to reproduce a traumatic brain injury in rats that were infused through an implanted osmotic minipump. The administration of the DHA-albumin complex resulted in a significant reduction in brain edema on both the damaged and contralateral hemispheres. In contrast, on the damaged hemisphere, albumin by itself did not show protection against FPI-induced brain edema. This ability to decrease the degree of edema could help reduce brain damage and subsequent learning disabilities after traumatic brain injuries.

### BRIEF DESCRIPTION OF THE DRAWINGS

FIG. 1 is a bar chart comparing total neuroscore of the identified treatment groups following MCAo. Repeated-measures ANOVA; *, different from all other groups, Dunnett's test; #, different from DHA-Alb 1.25 and from Alb 0.63 groups, Bonferroni test (p<0.05).

FIG. 2 is a bar chart comparing cortical infarct areas and volume of the identified treatment groups following MCAo. Repeated-measures ANOVA; *, different from saline group, Dunnett's test (p<0.05).

FIG. 3 is a bar chart comparing striatal infarct areas and volume of the identified treatment groups following MCAo. Repeated-measures ANOVA; *, different from saline group, Dunnett's test (p<0.05).

FIG. 4 is a bar chart comparing total infarct areas and volume of the identified treatment groups following MCAo. Repeated-measures ANOVA; *, different from saline group, Dunnett's test (p<0.05).

FIG. 5 is a bar chart comparing the neuroprotective effect of DHA-albumin complex with albumin expressed as a fraction of saline group. No significant differences between the two groups (Student t-tests).

FIG. 6 illustrates the change in the permeability of the blood brain barrier as determined by Evans blue extravasation into the brain in rats undergoing various treatments, including naïve rats, rats infused with saline after a sham operation, rats infused with saline after a traumatic brain injury, rats infused with albumin alone after a traumatic brain injury, and rats infused with the DHA-albumin complex after a traumatic brain injury.

### DETAILED DESCRIPTION OF THE INVENTION

The present invention relates to compositions comprising isolated complexes of polyunsaturated fatty acid (PUFA), particularly DHA, and albumin. The term “isolated” means the complex of PUFA and albumin is not in its natural state (e.g. not in the human body). The albumin used to obtain the complex can be either human serum albumin (hAlb) or recombinant albumin. The PUFA-Albumin complex may comprise more than one polyunsaturated fatty acid, as well as monounsaturated and saturated fatty acids. The complex is administered parenterally as soon as practicable after the onset of an acute brain insult resulting from ischemic stroke or traumatic injury and preferably within four hours of the insult. The complex can also be administered prophylatically during surgical procedures wherein the potential for ischemic tissue damage is present.

Pharmaceutical formulations for parenteral administration include, for instance, aqueous solutions of PUFA-Albumin complex or other appropriate suspensions. The pharmaceutical formulations are administered in a therapeutically effective dose, which refers to that amount of the complex that results in a reduction in the otherwise expected severity of ischemic or hemorrhagic tissue damage. The preferred dose for humans ranges between about 0.25 to about 2.5 grams (on an albumin weight basis) of PUFA—laden albumin per kilogram of body weight.

The present invention arose from a series of laboratory investigations wherein the effect of a DHA-Albumin complex was compared to treatment of albumin alone after an induced temporary MCAo. Animals were randomly assigned to 1 of 5 treatment groups: (1) Albumin, 1.25 g/kg (n=10); (2) DHA-Albumin, 1.25 g/kg (n=7); (3) Albumin, 0.63 g/kg (n=7); (4) DHA-Albumin, 0.63 g/kg (n=7); and (5) Normal saline (n=8).

The DHA-Albumin complex used in testing was prepared from the following protocol: Five vials containing 20 ml of human serum albumin (25%) were incubated with 4.0 mg DHA/g hAlb (molar ratio=0.2); incubation was performed in a shaking incubator at 37 degrees Centigrade for 30 minutes with vortex mixing every 5 min; aliquots (100 μl) from each vial were extracted and free fatty acids (FFA) were isolated by thin-layer chromatography (TLC), derivatized to fatty acid methyl esters (FAME) and analyzed by gas liquid chromatography (GLC); each vial was aliquoted in 5 ml samples and kept under nitrogen in a cold room for two months; and vials were gassed with nitrogen every week. There was no significant change in DHA and other polyunsaturated fatty acids loads on the hALb. The results show that the DHA-Albumin complex is stable with an expected product shelf life of at least about 4 to 6 weeks.

The DHA-Albumin complex in each of the five samples was analyzed to determine the amount of DHA loaded onto the albumin. The following tables illustrate the effect of the DHA incubation.

Fatty Acid Concentration (nmol/ml albumin)

| Fatty Acid | Sample 1 | Sample 2 | Sample 3 | Sample 4 | Sample 5 |
|---|---|---|---|---|---|
| 16:0 | 274.2 | 275.1 | 307.0 | 266.9 | 256.7 |
| 18:0 | 71.3 | 71.1 | 79.3 | 66.5 | 65.6 |
| 16:1 | 34.0 | 33.7 | 37.1 | 32.3 | 32.7 |
| 18:1 | 380.2 | 384.3 | 439.3 | 378.8 | 356.0 |
| 18:2n-6 | 230.2 | 191.0 | 247.9 | 213.2 | 201.2 |
| 20:4n-6 | 16.7 | 14.7 | 16.6 | 14.4 | 13.2 |
| 18:3n-3 | 13.7 | 12.9 | 14.9 | 13.7 | 12.1 |
| 20:5n-3 | 1.3 | 1.4 | 1.1 | 1.0 | 1.0 |
| 22:5n-3 | 8.0 | 8.4 | 9.4 | 8.0 | 6.4 |
| 22:6n-3 | 2005.6 | 2028.4 | 2219.8 | 2040.0 | 1589.3 |
| TOTAL: | 3035 | 3021 | 3372 | 3035 | 2534 |
| mg DHA/g | 2.64 | 2.67 | 2.92 | 2.68 | 2.09 |
| hAlb | | | | | |

Note that less than 4.0 mg DHA/g hAlb was incubated in Sample 5 resulting in a lower DHA load.

After preparing the DHA-Albumin complex, Male Sprague-Dawley rats (260-357 g) were anesthetized with halothane and nitrous oxide and subjected to up to 120 minutes of temporary MCAo by retrograde insertion of an intraluminal nylon suture coated with poly-L-lysine through the external carotid artery into the internal carotid artery and middle cerebral artery. Temperature probes were inserted in the rectum and the left temporalis muscle. Heating lamps were used to maintain rectal and temporalis muscle temperatures at 36 to 37 degrees Centigrade. In all rats, polyethylene catheters were introduced into the right femoral artery and vein for blood pressure recording, blood sampling, and drug infusion. Mean arterial blood pressure (MABP), plasma glucose, blood gases and hematocrit were continuously measured during the procedure.

Behavioral tests were performed in all 39 rats before MCAo, during occlusion at 60 minutes, and after treatment at 1 hour, 24 hours, 48 hours, and 72 hours. The battery consisted of two standardized tests used to evaluate various aspects of neurologic function: (1) the postural reflex test, which is used to examine upper body posture while the animal is suspended by the tail; and (2) the forelimb placing test, to examine sensorimotor integration in forelimb placing responses to visual, tactile and proprioceptive stimuli. Neurological function was graded on a scale of 0-12 (normal score=0, maximal score=12).

The drug (DHA-albumin, 1.25 or 0.63 g/kg; human albumin, 25% solution, 1.25 or 0.63 g/kg; or normal saline) was administered intravenously at the time of reperfusion, i.e., 2 hours from the onset of MCAo. The animals were allowed to survive for three days. Brains were then perfusion-fixed with a mixture of 40% formaldehyde, glacial acetic acid and methanol (FAM, 1:1:8 by volume), and infarct volumes and brain swelling were determined at 9 coronal levels throughout the brain. Repeated-measures ANOVA with post-hoc Bonferroni tests were used to assess infarct areas. Bonferroni-corrected Student t-tests were used in the non-repeated-measures comparisons. P<0.05 was regarded as significant.

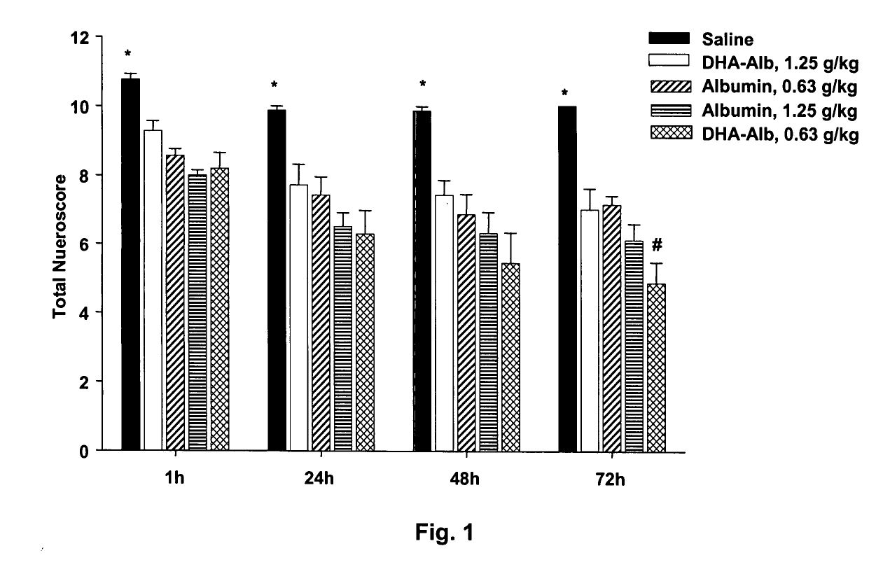

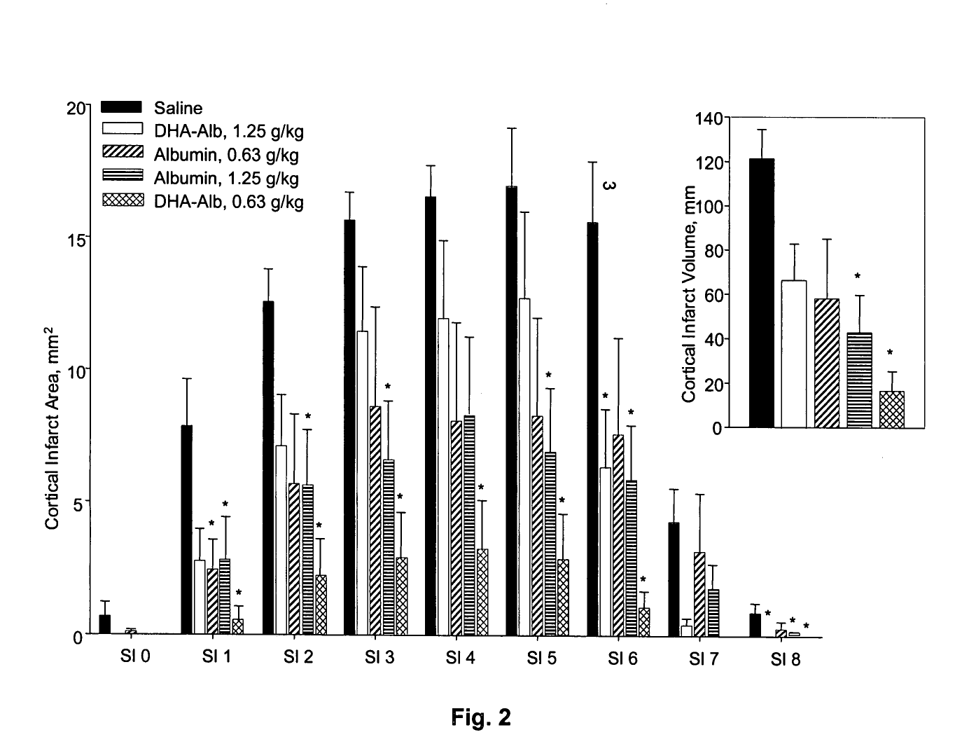

Physiological variables were stable and showed no significant differences among treatment groups. As can be seen in FIG. 1, while all treatments improved total neuroscore compared to saline, DHA-Albumin, 0.63 g/kg treatment reduced the 72 hr neuroscore significantly more than Albumin, 1.25 g/kg. FIG. 2 illustrates that DHA-Albumin, 0.63 g/kg treatment reduced cortical infarct areas at multiple coronal levels and it markedly reduced integrated cortical infarct volume by approximately 85%. While Albumin, 1.25 g/kg treatment also showed high-grade neuroprotection, the DHA-Albumin, 0.63 g/kg treatment group tended to be more highly protective (p=NS, i.e., >0.05).

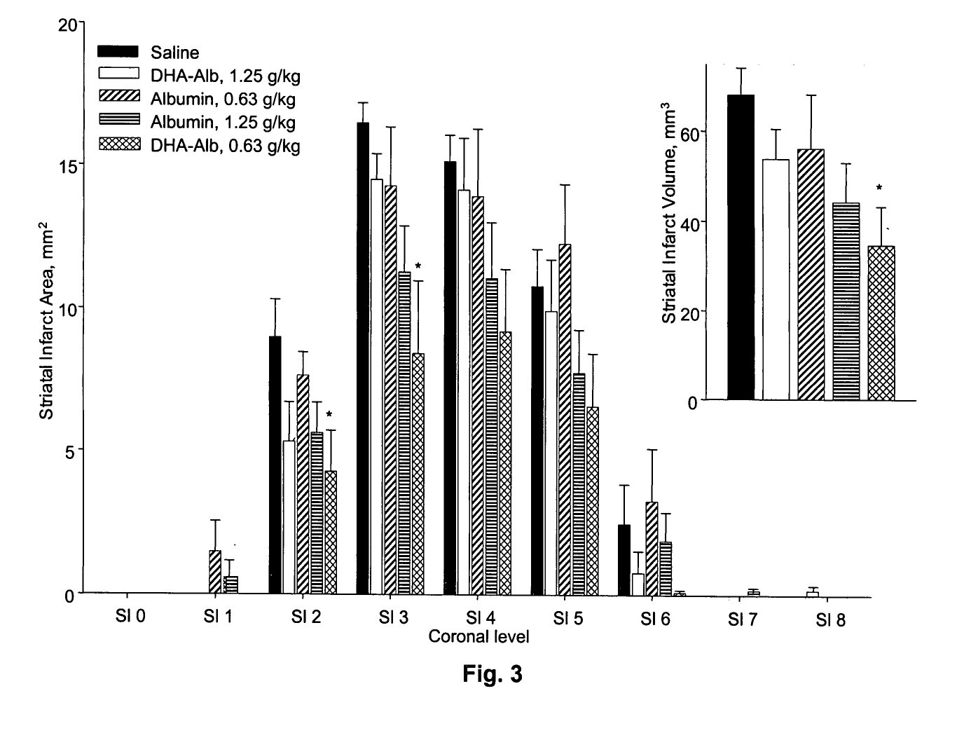

As shown in FIG. 3, the size of the subcortical infarct was reduced at 2 levels by DHA-Albumin, 0.63 g/kg treatment, and integrated striatal infarct volume was significantly reduced. Albumin alone was not able to achieve this effect.

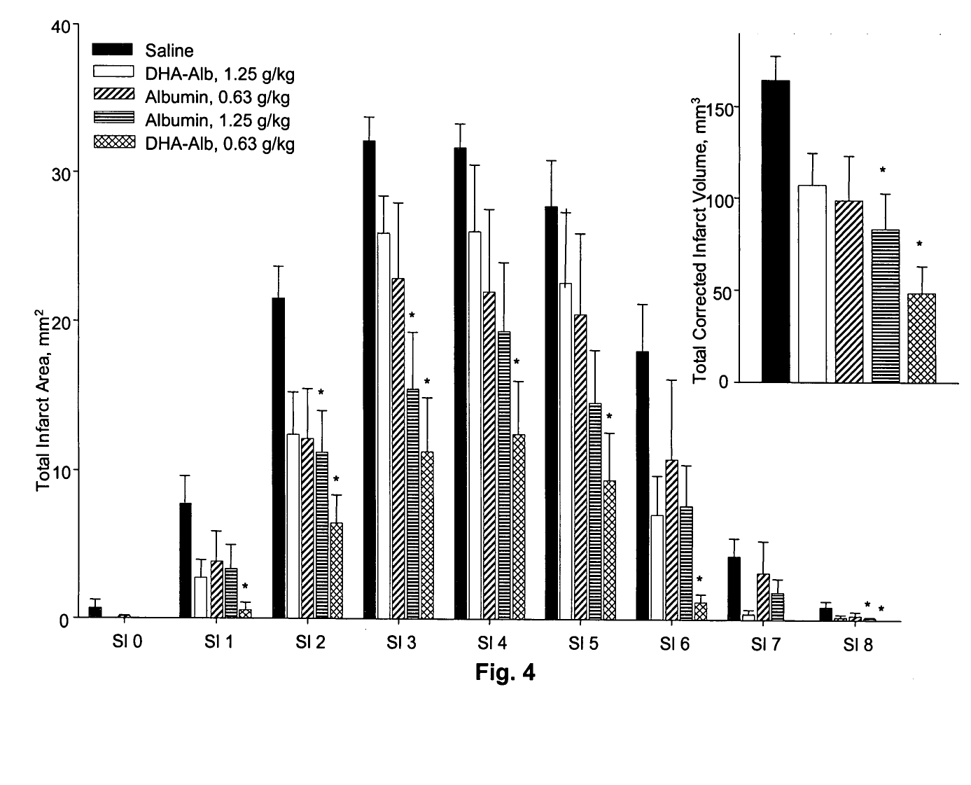

In FIG. 4, the total (cortical+subcortical) infarct was protected by DHA-Albumin, 0.63 g/kg treatment at multiple coronal levels, and the total (edema-corrected) infarct volume was reduced by approximately 70%. This degree of neuroprotection tended to be greater than the protection conferred by Albumin, 1.25 g/kg treatment, although this comparison was not statistically significant.

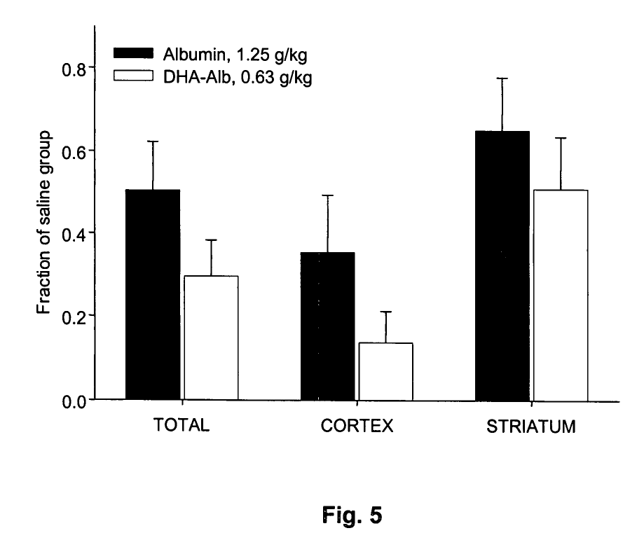

Finally, as shown in FIG. 5, the statistical comparison of DHA-Albumin, 0.63 g/kg treatment and Albumin, 1.25 g/kg treatment revealed no significant differences. This underscores the fact that DHA-Albumin, 0.63 g/kg treatment is as fully neuroprotective as treatment with albumin, 1.25 g/kg; in other words, when DHA is added to albumin, a high-grade neuroprotective effect is achieved at lower albumin doses.

In addition to reduced infarct areas, DHA-Albumin complex exhibited a dramatic decrease in brain swelling as estimated by the wet weight/dry weight method after cerebral injury. The following table illustrates the efficacy of DHA-Albumin complex to reduce brain swelling.

| | |
|---|---|
| Saline | 8.9 ± 5.2% |
| Albumin, 1.25 g/kg | 9.2 ± 3.9% |
| DHA-Albumin, 1.25 g/kg | 7.9 ± 4.0% |
| Albumin, 0.63 g/kg | 9.4 ± 7.0% |
| DHA-Albumin, 0.63 g/kg | 5.5 ± 2.4% |

* p = 0.004 vs. saline, Student t-test

Based on these results, the DHA-Albumin complex is an effective neuroprotective agent that can be used in treating ischemic stroke, injuries that may produce ischemic or traumatic tissue damage, and for reducing the potential for ischemic tissue damage during surgical procedures.

For use in the fluid percussion head injury model, DHA was physically complexed to human albumin by the following method. The complex was prepared under sterile conditions in a laminar-flow hood. DHA (200 mg cis-4,7,10,13,16,19-Docosahexaenoic acid, sodium salt; #D-8768, Sigma Co., St. Louis, Mo.) was dissolved in 500 μl ethanol, and then injected into a 100-ml sealed bottle of Buminate 25% (human serum albumin, USP, 25% solution; Baxter Healthcare Corporation, Westlake Village, Calif.). The solution was thoroughly mixed at room temperature in a G24 environmental incubator shaker (New Brunswick Scientific Co., Edison, N.J.) at 500 rpm for 30 min. Quantitative analysis by mass spectrometry and gas-liquid chromatography indicated 0.999 μg DHA/μl solution. The solution was stored at 4° C. protected from light and oxygen (stored under nitrogen). The solution was stable for at least six months.

Wilstar rats (250-300 g) were commercially obtained and housed at Louisiana State University Health Sciences Center, Neuroscience Center Animal Care Facilities in accordance with National Institutes of Health guidelines. Protocols were approved by the Louisiana State University Health Sciences Center Institutional Animal Care and Use Committee (IACUC). The rats were anesthetized with halothane, and a 4 mm craniotomy was done on the right parietal cortex. A 2 mm female union-bolt was cemented to the skull. Animals were allowed to recover for 24 hr. A fluid percussion injury to the right side was then done using 3 atm for 100 ms duration similar to that described in R. Vink et al., “Small shifts in craniotomy position in the lateral fluid percussion injury model are associated with differential lesion development,” J. Neurotrauma, vol. 18, pp. 839-47 (2001) and X. Q. Yuan et al., “The effects of traumatic brain injury on regional cerebral blood flow in rats,” J. Neurotrauma, vol. 5, pp. 289-301 (1988).

Rats subjected to the FPI procedure as described above were found to have impaired short-term and long-term memory impairment, as measured by the Morris water maze. Also, the blood brain barrier (BBB) permeability was assessed by measuring Evans blue dye extravasation into the brain, similar to the method described in R. Noor et al., “Hyperthermia masks the neuroprotective effects of tissue plasminogen activator,” Stroke, vol. 36, pp. 665-669 (2005). BBB permeability was found to increase in the damaged hemisphere (right) by 2.3-, 1.9- and 1.5-fold at 24, 48, and 72 h, respectively, as compared with sham animals (animals that underwent the same operation but without the injury). In the contralateral hemisphere a significant alteration of the BBB permeability was observed only at 24 h (a 2.4-fold increase over sham animals), indicating a faster recovery than the damaged side. In a second experiment, rats were implanted with minipumps intraperitoneally. Mini-osmotic pumps (Alzet-model 1007D), were prepared and filled with either saline, the DHA-human serum albumin (HSA) complex, or with only HSA. The pumps were inserted in the intraperitoneal cavity of each rat, and an infusion rate of 6.72 ug/kg/day.

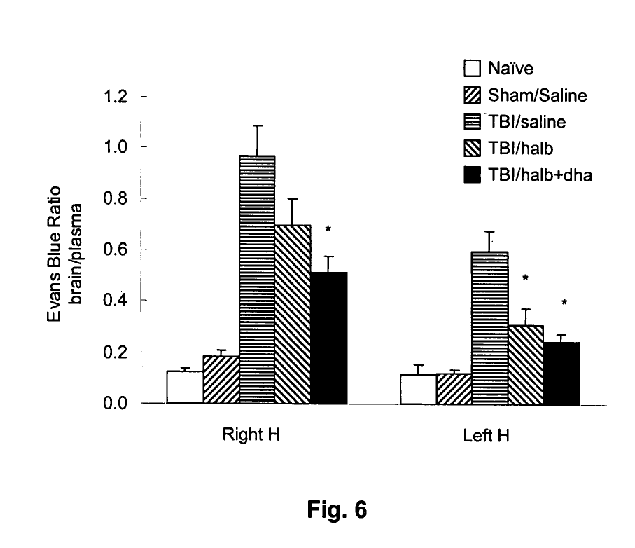

The results are shown in FIG. 6, where the bars represent mean values±SEM, and the number of rats in each study is from 8 to 11. The Evans Blue ratio was calculated based on (ng EB/g brain tissue)/(μg EB/g plasma). The values that were significantly different from the TBI/saline group are indicated by an “*” based on a Student's t test, p<0.05. The rats that were continuously infused with DHA-Halb (0.63 g/kg) showed at 24 h post-FPI a significant reduction in brain edema in both the right (damaged side) and left hemispheres, reductions of 47% and 59%, respectively (p<0.05). (FIG. 6) In contrast, the infusion of only Halb showed a tendency to lower the Evans blue estravasation into the right damaged hemisphere, but the difference was not significant (reduction of 28%, p>0.05). In the contralateral hemisphere, Halb reduced edema by a significant amount, 48%, but was still less than the reduction seen with DHA-Halb (59%).

The infusion of DHA-Halb protected the brain from FPI-induced alterations in both the damaged and contralateral hemispheres. Vasogenic brain edema is the more common cause of mortality after a severe head injury.

The term “therapeutically effective amount” as used herein refers to an amount of the DHA-albumin complex sufficient to decrease the amount of edema due to a traumatic head injury to a statistically significant degree (p<0.05). The term “therapeutically effective amount” therefore includes, for example, an amount sufficient to decrease the permeability of the blood brain barrier by at least 30%, and more preferably by at least 70%. The dosage ranges for the administration of DHA-albumin are those that produce the desired effect. Generally, the dosage will vary with the age, weight, condition, and sex of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the BBB permeability by methods well known to those in the field and by methods taught by this Specification. Moreover, the DHA-albumin complex can be applied in pharmaceutically acceptable carriers known in the art. The application is preferably by injection or infusion. If given orally, the DHA-albumin complex would preferably be protected from digestion.

The DHA-albumin complex may be administered to a patient by any suitable means, especially parenterally or vascularly. Parenteral infusions include intramuscular, intravenous, intraarterial, intraperitoneal or intravitreal administration. Additionally, the infusion could be directly into an organ, e.g., the brain. Injection of DHA-albumin may include the above infusions or may include intraperitoneal, intravitreal, direct injection into a blood vessel or into the cerebral spinal fluid. The DHA-albumin complex may also be administered transdermally, for example in the form of a slow-release subcutaneous implant, or orally in the form of capsules, powders, or granules. Although direct oral administration may cause some loss of activity, the DHA-albumin complex could be packaged in such a way to protect the active ingredient(s) from digestion by use of enteric coatings, capsules or other methods known in the art.

The present invention provides a method of inhibiting or attenuating the damage to the brain following a traumatic brain injury, in particular decreasing the amount of brain edema, comprising administering to a patient who has suffered a traumatic brain injury a therapeutically effective amount of DHA-albumin complex. The term “attenuate” refers to a decrease or lessening of the symptoms of brain edema.

Although the present invention has been described in terms of specific embodiments, it is anticipated that alterations and modifications thereof will no doubt become apparent to those skilled in the art. It is therefore intended that the following claims be interpreted as covering all alterations and modifications that fall within the true spirit and scope of the invention.

We claim:

1. A method to inhibit or attenuate symptoms associated with a traumatic brain injury, comprising administering a therapeutically effective dose of a pharmaceutical composition comprising albumin and a polyunsaturated fatty acid.

2. The method of claim 1 wherein the albumin is human serum albumin.

3. The method of claim 1 wherein the albumin is recombinant human albumin.

4. The method of claim 1 wherein the polyunsaturated fatty acid is docosahexaenoic acid.

5. The method of claim 1 wherein the pharmaceutical composition is prepared by forming an albumin-polyunsaturated fatty acid complex by incubating albumin in the presence of docosahexaenoic acid such that the docosahexaenoic acid physically binds to the albumin forming an albumin-docosahexaenoic acid complex.

6. The method of claim 5 wherein the therapeutically effective dose comprises from about 0.25 grams per kilogram of bodyweight to about 2.5 grams per kilogram of bodyweight of the albumin-docosahexaenoic acid complex on an albumin weight basis.

7. The method of claim 5 wherein the therapeutically effective dose comprises from about 0.5 milligrams per kilogram of bodyweight to about 7.5 milligrams per kilogram of bodyweight of the albumin-docosahexaenoic acid complex on an docosahexaenoic acid weight basis.
